(19) United States
(12) Patent Application Publication
(10) Pub. No.: US 2005/0063979 A1
(43) Pub. Date: Mar. 24, 2005

(54) Antigen presenting vesicles

(76) Inventors: Winfried Pickl, Vienna (AT); Brian Seed, Boston, MA (US); Sophia Derdak, Vienna (AT)

Correspondence Address: CLARK & ELBING LLP, 101 FEDERAL STREET, BOSTON, MA, 02110, US

(21) Appl. No.: 10/492,067

(22) PCT Filed: Nov. 6, 2002
(86) PCT No.: PCT/IB02/04637

(30) Foreign Application Priority Data
Nov. 6, 2001 (EP) 011263431

(51) Int. Cl. A61K 39/00; C12N 1/00

(52) U.S. Cl. 424/184.1; 435/317.1

(57) ABSTRACT

The present invention relates to an antigen presenting membrane vesicle comprising on its surface a composition of either relevant molecules for antigen-specific activation or deactivation of T lymphocytes and which is present in the form of an artificially induced lipid vesicle budded from a plasma membrane of a eukaryotic, preferably human, cell, and wherein the composition of said relevant molecules for activation or deactivation present on the vesicle surface is adjustable to a recipient's needs or requirements independently of any blood or tissue cells of said recipient. The invention further relates to a method of manufacture of the vesicles and to compositions containing the vesicles as well as to the use of the vesicles for various purposes including medical and diagnostic applications.

### TECHNICAL FIELD

The present invention is mainly in the field of immunology and relates to antigen presenting membrane vesicles, particularly to subcellular antigen presenting vesicles (SAV), which are produced by eukaryotic cells upon transfection with viral gene sequences and, optionally, with immune receptors. The antigen presenting vesicles bear relevant molecules for first and, optionally, second signals for antigen-specific activation or deactivation (e.g. inhibition) of T lymphocytes. The invention further relates to a method for the manufacture of the vesicles, to compositions containing the vesicles and to methods of use.

### BACKGROUND OF THE INVENTION

It is known that dendritic cells, which are derived from monocytes or CD34⁺ stem cells, are extremely potent antigen presenting cells that are able to present tumor-associated or viral antigens to the immune system. Such cells are capable of inducing specific immunity against distinct forms of cancer or infectious diseases. In order to do so, they express cell surface molecules, which are able to activate naive T cells. However, it is a main disadvantage in medical practice that for therapeutic application these dendritic cells need to be produced in a great number under GMP (good manufacturing practice) conditions from a sample of the respective, histocompatible patient ex vivo.

Methods, in which cells become transfected with HLA molecules and T cell costimulatory molecules in order to generate artificial antigen presenting cells, are known to the expert (Latouche and Sadelain, 2000; Sprent et al., 1997). It has been shown that co-expression or transfection of adhesion molecules, such as ICAM-1, enhances the potential of antigen presenting cells to induce effector cells (Camacho et al., 2001). In contrast to the present invention which describes a sub-cellular, i.e. cell-free, antigen presenting system, the artificial antigen presenting systems known in the art represent cellular systems. Moreover, some of them such as the artificial antigen presenting cells described by Sprent et al., are even based on insect cells (Sprent et al., 1997).

### DESCRIPTION OF THE INVENTION

It is an object of the invention to produce an agent which independently of a patient's cells, e.g. tissue or blood cells, is able to exert the antigen presenting function similar to dendritic cells derived from peripheral blood or CD34⁺ stem cells. It is understood in the art that HLA molecules are cell surface proteins which are encoded on the human chromosome 6 and which, in the case of HLA class I molecules, consist of a polymorphic alpha chain and the monomorphic beta-2 microglobulin. In the case of class II molecules, they consist of a polymorphic alpha and a polymorphic beta chain. The HLA molecules bind protein fragments in the form of oligopeptides and present them to effector cells of the immune system, especially to T cells.

The term “costimulatory molecule” as used herein includes any molecule which is able to either enhance the stimulating effect of an antigen-specific primary T cell stimulant or to raise its activity beyond the threshold level required for cellular activation, resulting in activation of naive T cells. Such a costimulatory molecule can be a membrane-resident receptor. For the construction of of antigen presenting cells according to the present invention receptors such as CD80, CD86 and CD58, alone or in combination, are suitable.

The term “adhesion molecule” relates to molecules which enable, facilitate or strengthen a physical contact between two cells, e.g. immune cells, thus allowing or improving further interactions between those cells. Adhesion molecules are usually cell surface receptors. In the interaction of an antigen presenting cell (APC) with a T lymphocyte there are different adhesion molecules involved such as, e.g., CD54 (ICAM-1) or DC-SIGN (CD209).

The term “antigenic peptide” primarily relates to proteolytic fragments of foreign proteins or tumor proteins that have been generated in the cytosole of human cells by the proteolytic action of so-called proteasomes, and that have been actively transferred into the endoplasmatic reticulum by transporters. Due to their primary amino acid sequence, only those foreign peptides or tumor peptides exert antigenic activity, which are bound by the so-called antigen-binding groove of HLA molecules. Examples for such antigenic peptides comprise viral antigens, tumor associated antigens, but also allergens and autoantigens.

The combination of relevant representatives of each of the four above-described groups of molecules, i.e. HLA molecules, costimulatory molecules, adhesion molecules, and antigenic peptides, on a single cell allows to design an antigen presenting cell with either specific T cell activating or specific T cell inhibitory potential, as desired. It is an object of the present invention to provide an antigen presenting unit in the form of a cell free system that is adapted in a way such that HLA molecules, costimulatory molecules, adhesion molecules and antigenic peptides are active in a concentrated manner and in a concerted mode of action towards, e.g., human T lymphocytes. It is a particular advantage of the present invention that the composition of the individual components described above can be freely varied, preferably to meet different immunological, histological and/or physiological needs or requirements of application and particularly of a recipient in need of or benefitting from a treatment with the present vesicles.

According to the invention subcellular antigen presenting vesicles (SAV), which carry the molecules required for antigen presentation and T-lymphocyte activation or inhibition (anergization) on their surface, are used for that purpose. The present SAV in analogy to antigen presenting dendritic cells thus contain the most important elements which are required for the formation and maintenance of the so-called immunological synapse between APC and T lymphocyte.

It is known in the art that there are natural forms of vesicle formation by eukaryotic cells, e.g. the formation of exosomes (Johnstone and Ahn, 1990). Furthermore, it is known that dendritic cells secrete subcellular vesicles which contain distinct cell surface molecules of the secreting dendritic cells (Thery et al, 2001; Zitvogel et al., 1998).

In contrast to the known forms of antigenic vesicles, the present invention relates to vesicles which are produced upon artificial induction using genetic engineering techniques, which allows to generate broadly modifiable SAV. Although produced artificially, their activity is very similar to the natural mode of action of e.g. dendritic cells. Unlike the SAV of the present invention, some antigen presenting exosomes known in the art are constitutively produced from tumor cells or dendritic cells (Zitvogel et al. 1998, Quah et al., 2000, Clayton et al., 2001) and are derived from so called “multivesicular bodies” which are generated by the invagination of endo-lysosomal membranes (Denzer et al., 2000). Likewise, and in contrast to the present invention, the exosomes described by Raposb et al. are constitutively produced by culturing B-cells, macrophages or dendritic cells, which by nature have already strong antigen presenting potential (Raposo et al., 1996).

The production of enveloped viruses by infected cells results in the formation of vesicles which contain proportions of the plasma membrane. The so-called core proteins of retroviruses, e.g. of Moloney murine leukemia virus (Moloney MLV), are responsible for the budding process. These core proteins become concentrated in the so-called lipid rafts of the plasma membrane, which are cholesterol-, glycosphingolipid-, and ganglioside-enriched areas of the plasma membrane (Simons et al., 2000). Likewise, envelope proteins of phylogenetically very distinct RNA and DNA viruses become enriched in such areas of the plasma cell membrane, and also virus budding takes place in those areas of the plasma membrane (Pickl et al., 2001). In the last years it also became increasingly clear that immune cells use lipid rafts for the enrichment and display of important receptors and kinases which are relevant for signal transduction. Lipid raft affiliation favors protein-protein interactions in the context of signal transduction (Simons et al., 2000).

The present invention uses viral core sequences for induced vesicle formation. “Viral core sequences” are understood as viral, especially retroviral, core proteins or parts of core proteins such as, for example, the group-specific antigens (GAG) of. Moloney MLV or HIV or the M1 protein of influenza virus.

As has been shown recently, posttranslational lipid modification of viral or cellular proteins is responsible for the enrichment of envelope proteins in lipid rafts and thus in the envelope of enveloped viruses (Pickl et al., 2001). “Posttranslational lipid modification” describes the covalent attachment of one or several fatty acids, mostly unsaturated fatty acids, to the proteins to be modified within the endoplasmatic reticulum, which leads to preferential anchoring of such modified proteins in the lipid rafts of the plasma membrane (Melkonian et al., 1999; Pessin and Glaser, 1980). According to the present invention myristoylation, palmitoylation and glycosylphosphatidylinositol-(GPI)-anchorage are suitable methods for that purpose.

Lipid modified proteins as described above are also applied according to the present invention. They are preferably selected from the group consisting of the ectodomains of human CD80 (B7.1), CD86 (B7.2), CD54 (ICAM-1), CD58 (LFA-3), and HLA class I and II molecules of any allele. In addition, the subcellular antigen presenting vesicles of the instant invention contain the endogenous, constitutively expressed human GPI anchored molecules CD55 and CD59 derived from the 293T cell line (FIG. 2). Both CD55 and CD59 are complement regulatory molecules that may inhibit or prevent rapid degradation of the vesicles in the body.

It is preferred that the recombinant molecules or genes introduced to the 293T cells with the help of the pEAK12 expression vector be overexpressed. This is accomplished by the use of an elongation factor-1-alpha promoter and other mechanisms known in the art, such as, for instance, plasmid amplification via an SV40 origin of replication and provision of episomal stability which is conferred by an EBV origin of replication. The overexpression of gene products may also contribute to enriching surface receptors in lipid rafts. This circumstance appears to be especially important for those molecules of the present invention that are expressed in a non-lipid modified form (such as the type 2 membrane proteins like DC-SIGN (CD209)).

According to the present invention, the SAV not only allow to communicate “antigen presenting signals” to T cells but also allow to adjust the composition of the HLA molecules and antigenic peptides according to the needs or requirements of a particular application or of a patient and/or of a particular disease. In this context, the intensity (extent) of T cell activation is dependent on the absence or simultaneous presence of additional costimulatory molecules and adhesion molecules attached to the SAV. (FIG. 4). That is, omission of costimulatory and/or adhesion molecules renders the present SAV so-called anergizing SAV, i.e. T cell inhibitory SAV. Such SAV are also encompassed by the present invention and can, for instance, be selectively designed and adapted to present allergenic or autoantigenic peptides for prophylactic or therapeutic application in the treatment of allergies or autoimmune diseases.

The SAV of the present invention represent a cell-free non-replicative system which combines, in a simple co-transfection system, the advantages of synthetic immunogenic peptides and binding reagents with the advantages known from liposome technology. Due to the artificial induction of the pinching-off process of plasma membrane vesicles from suitable living, preferably human, cells it is possible to take advantage of the physical and biological characteristics of plasma membrane vesicles, especially of their ability to interact with plasma cell membranes of other cell types, an ability which has been optimized by the co-evolution of (enveloped) viral and mammalian biosystems in a very long and natural selection process.

In contrast to the present invention, some artificial T cell activation systems known in the art are based on the use of non-biologicav-biocompatible particles (magnetic beads) which have been coated with monoclonal antibodies directed against cell surface molecules. Such systems are not designed nor suitable for an in vivo application and are not antigen specific (Grosmair et al., 2000).

The SAV according to the present invention allow for various applications. They are, for instance, suitable:
for the use in the prophylaxis or treatment of many disorders of the human or animal body, such as immune disorders, malignancies, infectious diseases, and others. In particular, they are suitable as a therapeutic agent to treat, for instance, tumor-patients;
as an agent to induce an immune response against poorly immunogenic agents, such as for instance tumor cells or infectious agents, that do not normally trigger a proper immune response, if at all;
as a tool to screen for the presence of tumor cells in vivo and ex vivo;
in a modified form as an anergizing agent to shut off already ongoing, especially undesired, immune responses in auto-aggressive immune disorders;
as a handy tool for the, optionally functional, analysis of novel costimulatory molecules, adhesion molecules or other molecules of interest;
for the efficient expansion of cell types or tissues in vitro or in vivo. By way of example, it is pointed to the SAV facilitated expansion or differentiation of any cell type of the immune system, in particular of antigen-specific T or B lymphocytes, but also to the expansion of, for instance, pluripotent precursor cells or stem cells that are able to differentiate into any tissue or cell type of the human or animal body;
for the application of transmitter substances or growth factors which are enriched in or on the present SAV and which can be targeted to the desired site of action, preferably with the help of surface receptors;
as a basis for the design and manufacture of multivalent binding reagents in order to identify antigen-specific mammalian cells, especially human immune cells. Suitable procedures for the generation of T cell receptor (TCR) specific binding reagents, consisting of, preferably fluorochrome conjugated, complexed MHC molecules harbouring a specific peptide, are known in the art. The MHC molecules which represent the major components of such binding reagents, also called tetramers, have to be purified from expression systems and subsequently assembled to dimers or tetramers artificially.

In contrast to the methods known in the art, the present SAV, expressing specific MHC-molecules, can be used directly as specific binding reagents. The MHC molecules as the major players in the system will, if targeted to lipid rafts, assemble and be enriched spontaneously on the present SAV. They will also be equipped with adequate posttranslational modifications such as, for instance, sugar moieties, because they are produced in suitable, preferably human, eukaryotic cells. Loading with antigen will be achieved either by transfecting the respective antigen in the form of an expressable gene, e.g. as a peptide antigen minigene, into the producer cell or, alternatively, by pulsing the SAV with excess of peptide antigen externally.

The multivalent nature of virus-sized particles will drastically augment binding avidity of said SAV as compared to the tetramer technology based systems known in the art. To facilitate visualization of the present SAV, labelling procedures such as e.g. biotinylation or introduction of stable membrane dyes can be used. The introduction of additional molecules to the present SAV, such as, for instance, adhesion molecules, will stabilize even very low-affinity interactions and will make it possible to visualize clonal T cell populations expressing even very low-affinity T cell receptors. Such SAV, obtainable according to the present invention, may thus comprise suitable, self-assembling “super-tetramers”.

The present SAV may further be applicable:
as a platform to express a receptor of interest (other than a MHC molecule) at high density. Said SAV can in turn be used as a multivalent binding reagent in order to identify and characterize putative ligands, expecially cellular ligands, for that receptor;
as a platform to display interacting elements (stimulatory and/or inhibitory) for any cell type, in particular when more than one said element is required, and preferably when co-localization of said interacting element on one and the same surface is desired.
as a platform to display said interacting elements at high concentration;
as a diagnostic tool for the functional and/or phenotypical characterization of cells, in particular immune cells, derived from individuals with, for instance, suspected immunodeficiency or, for instance from individuals who have been immunized with specific agents and whose cells shall be immuno-monitored for vaccination-success.

The advantage of the present SAV, as compared to antigen presenting exosomes known in the art, lies in the fact that the present SAV emerge directly from the plasma cell membrane, and further that their molecular make-up can be actively and very flexibly modified as described above. This has not been described for exosomes so far. Because exosomes emerge from so-called multivesicular bodies, a similar modification of their molecular composition with the above-described methods does not appear to be feasible. In addition, the application of exosomes is strictly dependent on the histocompatibility type of the cell donor used for their production, whereas the present SAV can be ‘tailored to a patient’s needs and requirements.

### BRIEF DESCRIPTION OF THE FIGURES

FIG. 1a: Schematic depiction of the CD5L::CD3-scFᵥ::IgG₁₋ and CD5L::CD3-scFᵥ::CD14 expression cassette (segment located within the pEAK 12 expression vector). The figure shows the individual genes and the restriction enzyme sites which have been used for the cloning and construction of the individual parts of the construct.

FIG. 1b: Schematic depiction of the expression cassette which has been used for the expression of the GPI-modified, type I transmembrane proteins (insert within pEAK12 expression vector). The expression cassette is used to modify cell surface receptors in order to enrich their concentration in lipid rafts. The figure shows the insert only in its form in the expression vector pEAK12.

FIG. 1c: Schematic depiction of the expression cassette for the OPG/OG constructs. The restriction sites which have been used in order to construct the GAG-POL and GAG versions of MOMLV within the MOMLV genome are shown. The picture shows the insert without the vector sequences.

FIG. 1d: Analysis of stable transfectants by flow cytometry. The figure shows the expression levels of the CD5L::CD3scFv::CD14 transgene in 293T cells (clone #19). The picture shows an overlay histogram of a typical flow cytometric experiment, showing the negative control at the left-hand side of the histogram and the staining pattern of the transgene on the right hand side of the histogram.

FIG. 2: Results of a Western Blot analysis showing the composition of subcellular antigen presenting vesicles.
lanes 17-23: samples from mock-transfected 293T CD5L::CD3::scFv::CD14 #19 cells.
lanes 26-32: samples from 293T CD3::scFv::CD14 #19 cells upon co-transfection with CD80, CD54, DC-SIGN and OGP.
used antibodies:
lane 17, 26: anti gag p30
lane 18, 27: anti CD14
lane 19, 28: anti flu-Tag
lane 20, 29: anti CD54
lane 21, 30: anti CD99
lane 22, 31: anti CD59
lane 23, 32: anti HLA class I

FIG. 3a: Scheme of an ELISA for the detection of distinct cell surface receptor molecules on SAV.

FIG. 3b: Results of an ELISA with SAV as the sample according to FIG. 3a; the optical density at 405 nm was determined at different time points after the addition of p-nitrophenylposphate as the substrate for the alkaline phosphatase.

FIG. 4: Proliferation of PBMNC upon stimulation with SAV which have been transfected with the indicated constructs. The figure shows counts per minute (cpm) after three days of culture and a 18 hour pulse with methyl-3H-thymidine.

The following examples shall explain the present invention in more detail and are not to be construed as limiting the invention in any respect.

### Example 1

### Production of a Subcellular Antigen Presenting Vesicle (SAV)

A procedure for the generation of said SAV includes the following steps:

a) Construction of CD5L::CD3-scFᵥ::CD14.

As a starting material we used a hybridoma cell line, which secreted the anti-human CD3 epsilon mAb OKT3 (American type culture collection). With the help of the salting out procedure we isolated genomic DNA from the hybridoma cell line. The genomic DNA served as a template for the synthetic amplification of DNA fragments encoding the hypervariable regions of the rearranged immunoglobulin VH and VL gene-stretches. For that purpose we amplified the respective DNA regions with the following primers (amplification conditions were chosen as follows: 25 cycles 94° C. 10 sec. 55° C. 10 sec. 72° C. 15 sec):

| | |
|---|---|
| VH: for | |
| GGAATTCGCTAGCCCAGGTCCAGCTGCAG | (SEQ ID NO 1) |
| CAGTCT, | |
| rev | |
| GGGGGATCCGGTGACCGTGGTGCCTTGGC | (SEQ ID NO 2) |
| CCCAGTA; | |
| VL: for | |
| GGAATTCGAGCTCCCAAATTGTTCTCACC | (SEQ ID NO 3) |
| CAGTCTCCA, | |
| rev | |
| GGGATCCCCACCGCCCCGGTTTATTTCCA | (SEQ ID NO 4) |
| ACTTTGTCCC. | |

The primers were designed in a way such that in the VH region at the 5′-end a Nhe I restriction site at the 3′-end a Bst EII site was placed. VL was flanked at the 5′-end by a Sac I site at the 3′-site by a Bam HI site. Upon digestion with the respective restriction enzymes the two amplified segment were cloned into the pBlueskript II KS+ vector, the sequence identity of the respective inserts was subsequently verified by nucleotide sequencing. Individual clones carrying the desired gene of interest were digested with the above mentioned restriction enzymes and cloned together with a (G₃S)₄ linker, which is flanked by Sac I and Bst EII restriction sites into the vector pBlueskript CD5Lneg1. The resulting construct was named CD5L::CD3-scFᵥ::neg1.

pBlueskript CD5Lneg1 contains in its 5′-end a leader sequence derived from the human CD5 gene as well as a human immunoglobulin G₁ sequence that codes for the hinge and the CH2 and CH3 constant regions of human IgG. At the 3′-end of the leader sequence two restriction sites, Bam HI and a Nhe I respectively, are located which were used for the insertion of the single chain antibody in-between the CD5 leader and the IgG1 gene.

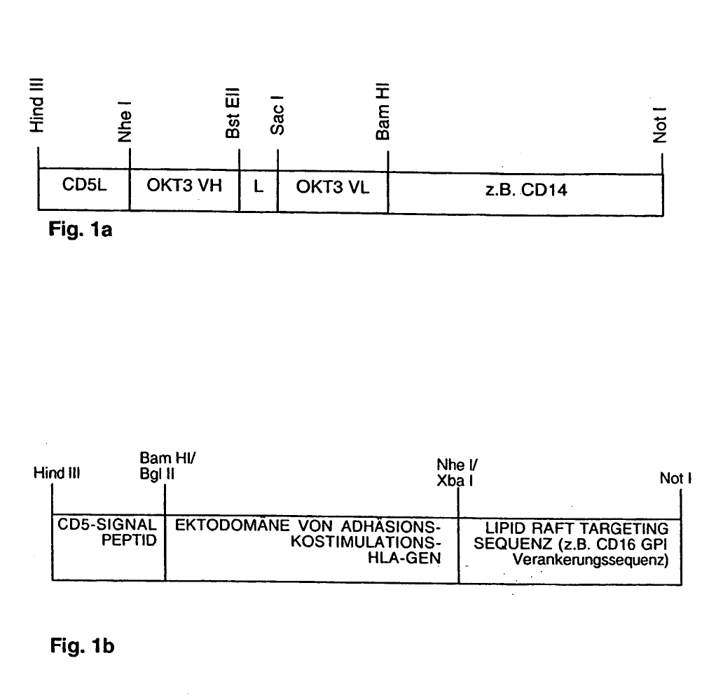

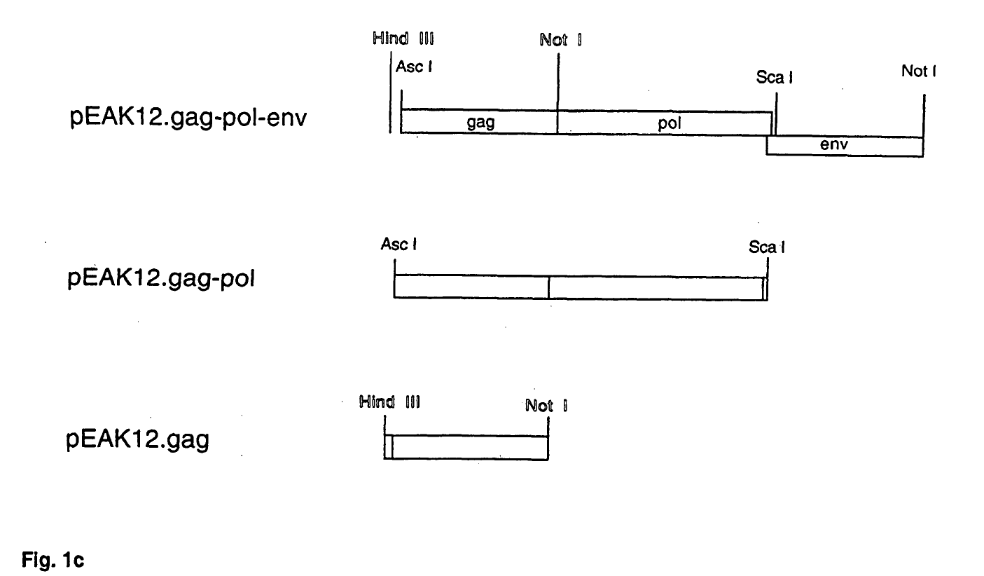

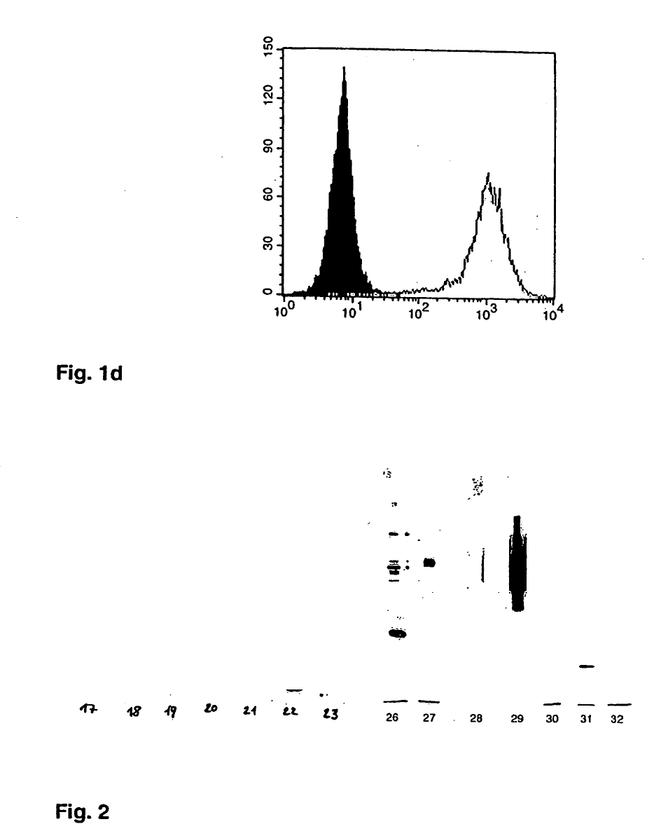

For eukaryotic expression we swapped the above described construct in the pEAK12 vector and termed the plasmid pEAK12 CD5L::CD3-scFᵥ::IgG₁ thereafter. Introduction of this plasmid leads to the production of a human IgG1 Fc-carrying antibody with anti-human CD3 specificity (FIG. 1a).

In order to generate a membrane-bound and lipid raft-targeted expression of the single-chain antibody, the IgG1 (hinge-CH2-CH3) sequences were replaced by the ectodomain of the CD14 molecule, which has been PCR amplified and tagged with Bam HI and Not I restriction enzyme sites at their ends. The construct when expressed in pEAK12 was termed pEAK12 CD5L::CD3-scFᵥ::CD14.

b) Expression of pEAK12 CD5L::CD3-scFᵥ::CD14 and pEAK12 CD5L::CD3-scFᵥ::IgG₁.

293T cells (4.5 10⁶ cells) have been transfected with 20 μg DNA according to the modified calcium phosphate transfection method as described (Jordan et al., 1996) in a 10 cm petri dish. A typical experiment used 2 ml of transfection mix which was in a typical experiment added to the petri dish containing 15 ml of IMDM cell culture medium supplemented with 10% fetal calf serum. 16 hours after the addition of the transfection mix the medium was replaced by 15 ml of fresh medium, subsequently cells were cultured for a further period of two (pEAK12 CD5L::CD3-scFᵥ::CD14) or nine (pEAK12 CD5L::CD3-scFᵥ::IgG₁) days respectively.

Stable expression of pEAK12 CD5L::CD3-scFᵥ::CD14 was achieved by transfecting 293T cells with the Avr II linearised vector-backbone followed by selection with 1 μg/ml puromycine. 20 stable transfectants were cloned, clone. #19 was chosen and used for further experiments.

The cell surface expression level of the CD5L::CD3-scFᵥ::CD14 transgene was determined in cell surface staining experiments with an anti-CD14 monoclonal antibody the binding of which was detected by secondary fluorochrome conjugated anti-mouse Ig reagents (FIG. 10).

The functionality (i.e. antigen specificity) of the OKT3 single chain antibody (CD5L::CD3-scFᵥ::IgG₁) was determined by the production-of a soluble form of the antibody, and the demonstration of its exclusive binding to no other cell types than human T cells.
c) Construction of Costimulatory, Adhesion and HLA Molecules with a GPI-Anchor Sequence in pEAK12

The construction of CD5L::CD80::CD16 was designed in the following way: Starting from the respective cDNA sequences, which code for the individual genes, we amplified with the help of PCR technology the individual genes with modified-ends. The expression cassette used the following restriction sites: Hind III and Bam HI (for CD5L), Bam HI and Nhe I (for the cell surface receptor of interest), and Nhe I and Not I (for the GPI-anchor) (Abb. 1b).

Exemplary sequences of the oligonucleotides, which have been used for the generation of some of the ectodomain encoding constructs are listed in the following:

| | |
|---|---|
| CD80 for: | |
| CGCGGGGGATCCCGTTATCCACGTGACCAAGGA; | (SEQ ID NO 5) |
| CD80 rev: | |
| GCGCCGCTAGCGGATGGGAGCAGGTTATCAG; | (SEQ ID NO 6) |
| CD86 for: | |
| CGCGGGGGATCCCGCTCCTCTGAAGATTCAAGC; | (SEQ ID NO 7) |
| CD86 rev: | |
| GCGCCCGCTAGCGTGGTCTGGGGGAGGCTGAG; | (SEQ ID NO 8) |
| CD40 for: | |
| CGCGGGGGATCCCGAACCACCCACTGCATGCAG; | (SEQ ID NO 9) |
| CD40 rev: | |
| GCGCCCGCTAGCCACCAGGGCTCTCAGCCGAT; | (SEQ ID NO 10) |

The CD16-GPI anchoring sequence was amplified with the following oligonucleotides:

| | |
|---|---|
| CD16 for: | |
| CGCGGGGCTAGCTTGGCAGTGTCAACCATCTCA; | (SEQ ID NO 11) |
| CD16 rev: | |
| GCGCCCGCGGCGCTTTAAATGTTTCTCTTCACAG | (SEQ ID NO 12) |
| AGA; | |

The GPI anchoring sequence of CD59-GPI was amplified with the following oligonucleotides:

| | |
|---|---|
| CD59 for: | |
| GGATTCGCTAGCCTTGAAAATGGTGGGACATCC; | (SEQ ID NO 13) |
| CD59 rev: | |
| CCCGCGGCGGCCGCTTTAGGGATGAAGGCTCCAG | (SEQ ID NO 14) |
| GCTG. | |

HLA class I genes have been modified with the help of the following 30 oligonucleotides and have been moved to the expression cassette shown in FIG. 1b. Exemplary oligonucleotide sequences for the amplification of the HLA-A*02011 gene are:

| | |
|---|---|
| HLA-A*02011 for: | |
| CGCGGGGGATCCCTGACCCAGACCTGGGCG; | (SEQ ID NO 15) |
| HLA-A*02011 rev: | |
| CGCGGGGGATCCCCTGACCCAGACCTGGGCA; | (SEQ ID NO 16) |

We used either cloned cDNAs or cDNA libraries derived from PHA blasts, Raji or Jurkat cells or placenta tissue which have been generated in the laboratory of Dr. Brian Seed in Boston, Mass., USA for the generation of the respective constructs.

d) pEAK12.0G and pEAK12.0GP Constructs (FIG. 1c)

As a template for the generation of the GAG and GAG-POL expressing plasmides we used pEAK12.OGPE, which has been generated from pΨ-sequences as a Asc I/Not I fragment (Pickl et al., 2001). In order to replace the env gene we took advantage of the Sca I site at the 5′-end of MoMLV env and the synthetically added Not I site at the 3′-end of the env gene. After a filling-in reaction with the help of the Klenow fragment of DNA-Polymerase I the open ends were subsequently blunt-end ligated which resulted in the generation of pEAK12.OGP. The generation of pEAK12.OG was done by PCR with the help of a gag-specific reverse primer CGCGGGGCGGCCGCTTTAGTCATCTAGGGTCAGGAGGG (SEQ ID NO 17) and a pEAK12 specific forward primer CGACTCACTATAGGGAGAC (SEQ ID NO 18). The amplified product was digested with the Hind III and Not I restriction enzymes and inserted into the pEAK12 expression vector.
e) Generation of Expressable So-Called ‘Peptide Minigenes’ (Anderson et al., 1991).

Tumor and virus peptides of known HLA restriction are expressed as minigenes on the 3′-end of the highly efficient CD5 leader (signal sequence) in pEAK12. For that purpose and in order to increase the expression efficiency in human cells we used codon optimized synthetic oligonucleotide pairs which carry Bam HI and Not I compatible ends and are conveniently clonable into the CD5L carrying pEAK12 vector.

f) Transfection of Products from c) and d) into 293T CD5L::CD3-scFᵥ::CD14 Stable Transfectants and Harvesting of Supernatants

In first experiments we replaced the signal #1 of said vesicles with the CD5L::CD3-scFᵥ::CD14 fragment. The transfection of the costimulatory and adhesion molecules described in c) was performed with the modified calcium phosphate precipitation method according to Wurm et al. Likewise, the OGP construct described in d) was cotransfected in all instances at a constant level of 25% of the totally transfected DNA amount. 16 hours after the transfection the medium was changed completely and the cells were further cultivated for two additional days. Subsequently, the supernatants were harvested (15 ml per condition/plate) and cleared from particulate material by a centrifugation (670 g in a SORVALL RTH-250 rotor, 10 min, at room temperature) and a filtration (0.45 μm filter) step.

According to said invention the CD5L::CD3-scFᵥ::CD14 fragment was subsequently replaced by a HLA class I molecule (complexed with β₂microglobulin due to co-transfection of β₂m) and an immunogenic peptide for which the HLA molecule serves as a restriction element. For that purpose the 293T cell line was transfected with constructs expressing the respective HLA-molecule, β₂ microglobulin as well as with the peptide encoding plasmid (in similar ratios), suitable adhesion and costimulatory molecules as well as with the OGP construct. The SAV containing supernatants were produced and purified in a manner similar to the ones described above.

In the following the morphological and functional analyses as well as the use of SAV is described by further examples.

### Example 2

### Morphological Analysis of SAV

The immunogenic vesicle containing supernatants were ultra centrifuged (100000 g 30 in a Beckman SW-60 rotor, for 1 h, at 4° C.), whereafter the pellet was washed once in PBS followed by an additional centrifugation step as above, after which the pellet was solubilized in SDS-PAGE sample buffer and resolved by SDS-PAGE. The Western blots show that the transfected molecules became highly enriched in the SAV-containing pellet from the supernatants, ditto the constitutively expressed lipid raft resident molecules of the cell line were highly enriched in the SAV (FIG. 2). This confirms that the immunogenic supernatants contain particulate and pelletable material, i.e. subcellular vesicles, and that the SAV contain the molecules that had been targeted intentionally (hypothesis based) to the lipid rafts.

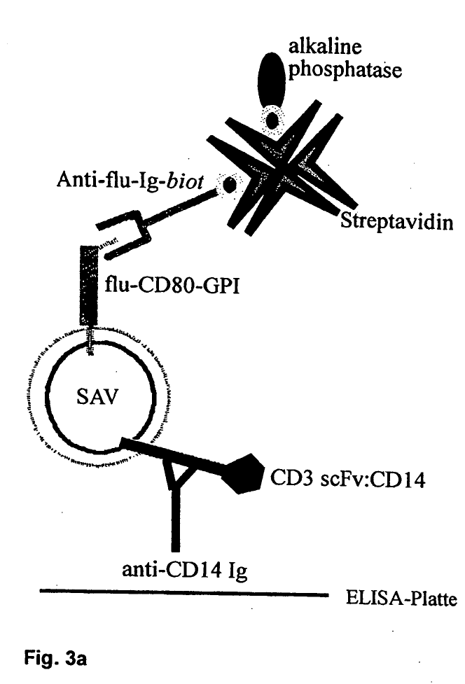

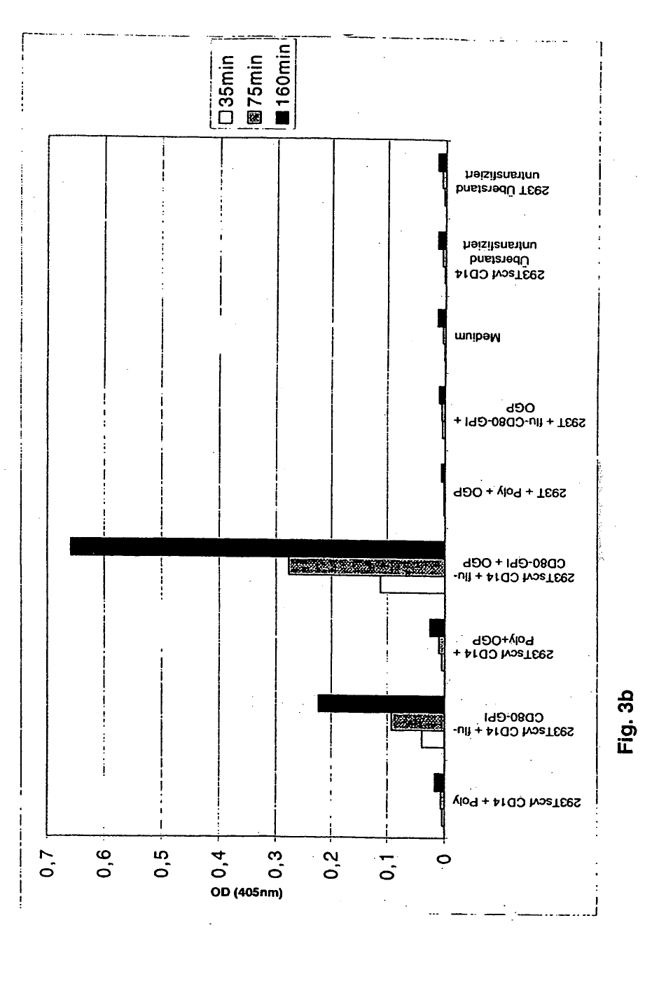

In addition, ELISA experiments demonstrated that the different transfected molecules are expressed and resident on one and the same particle (SAV) (FIG. 3).

The supernatants can be stored for four days at 4° C. or 37° C. without an alteration of their function.

Transmission electron microscopy studies confirmed that said SAV had the size of typical viral particles (data not shown).

### Example 3

### Functional Analysis of SAV

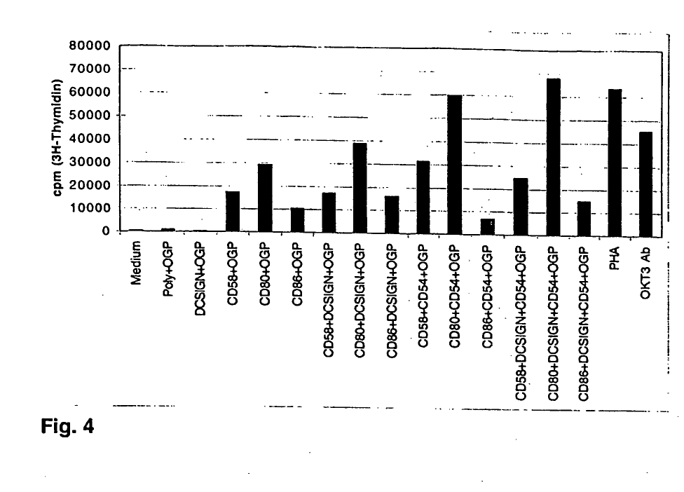

We tested the potential of said SAV containing vesicles for their effect on peripheral blood mononuclear cells derived from human blood (PBMNC). For that purpose we centrifuged 100.000 cells/well/100 μl together with (100 μl/well) SAV containing supernatants according to the spinoculation technology, which is broadly used for retroviral transduction of target cells, for 2 hours at 670 g in a SORVALL RTH-250 rotor at 30° C. Subsequently, cells were cultured for three days in a humidified atmosphere in a CO₂ incubator. The proliferation of the cells was then monitored for the following 18 hours by determination of the incorporation of ³H-thymidine into the newly synthesized DNA (FIG. 4).

Experiments performed with highly purified T lymphocytes demonstrated that our SAV stimulate T cells directly without the need for accessory cells (data not shown). In addition, experiments performed with SAV co-expressing PD-L2 along with CD80, CD54 and the surrogate TCR demonstrated that SAV with inhibitory receptor-combinations were successfully designed and obtained.

Cytotoxicity assays were chosen in order to investigate the cytotoxic potential of T cells that had been activated by SAV-containing supernatants. In those cases PBMNC of appropriate donors had been stimulated with SAV containing supernatants for seven days. Afterwards, cells had been restimulated in intervals of approximately ten days by the addition of cytokines (preferentially IL-2, IL-7 and/or IL-12) and freshly produced SAV, respectively.

As a read out we used HLA-typed target cells that had been labeled with ⁵¹chromium and which we co-cultured with graded amounts of the stimulated PBMNC for 4 h at 37° C. The supernatants of these reactions were collected and their degree of radioactivity was evaluated in a gamma-counter.

### Example 4

### Generation and Application of SAV as a Tumor Vaccine

According to the present invention, the herein-described SAV are designed for the application primarily in humans. For that purpose, the SAV may preferably be equipped with the above described adhesion and costimulatory molecules and with patient-specific (after determination of the histocompatibility type of the patient) HLA-molecules as well as compatible antigenic peptides, in order to achieve the desired activation or inhibition of effector cells.

To achieve this goal 293T cells can be cultured in serum-free or human-serum containing medium, whereafter, the SAV can be concentrated by an ultracentrifugation step (at 100.000 g for 1 hour at 4° C.). Alternatively, a sucrose-gradient may precede the concentration step in order to purify the SAV. The concentration of the SAV can be determined by the above-described capture ELISA with the help of anti-CD55 or anti-CD59 mAbs, which serves to capture the vesicles on the ELISA plates.

Administration of a pharmaceutical composition, particularly a cancer vaccine, comprising an effective dose of SAV prepared according to the present invention is accomplished by way of injection, preferably intradermal injection, to a cancer patient, or by means of a nasal spray.

### Example 5

### Generation and Application of Anergizing SAV

Anergizing SAV can be generated and applied as described in Example 4, with the exception that the SAV will solely express patient-specific HLA-molecules (after determination of the histocompatibility type of the patient) together with compatible allergens or autoantigens, where applicable, as a single component. Alternatively, combinations of adhesion and co-stimulatory molecules can be chosen, which, together with the patient-specific HLA-molecules as well as the compatible allergens or autoantigens, lead to the antigen-specific unresponsiveness of effector cells. Where appropriate, anergizing SAV can be generated which express patient-specific HLA-molecules as well as compatible allergens or autoantigens, together with such combinations of co-stimulatory and/or adhesion molecules that are able to activate suppressor cells of the immune system.

### REFERENCES

Anderson, K., Cresswell, P., Gammon, M., Hermes, J., Williamson, A., and Zweerink, H. (1991). Endogenously synthesized peptide with an endoplasmic reticulum signal sequence sensitizes antigen processing mutant cells to class I-restricted cell-mediated lysis, J Exp Med 174, 489-92.
Camacho, S. A., Heath, W. R., Carbone, F. R., Sarvetnick, N., LeBon, A., Karlsson, L., Peterson, P. A., and Webb, S. R. (2001). A key role for ICAM-1 in generating effector cells mediating inflammatory responses, Nat Immunol 2, 523-9.
Clayton, A., Court, J., Navabi, H., Adams, M., Mason, M. D., Hobot, J. A., Newman, G. R., Jasani, B. (2001). Analysis of antigen presenting cell derived exosomes, based on immuno-magnatic isolation and flow cytometry. J. Immunol. Meth. 247, 163-174.
Denzer, K., Kleijmeer, M. J., Heijnen, H. F., Stoorvogel, W., Geuze, H. J. (2000). Exosome: from internal vesicle of the multivesicular body to intercellular signaling device. J Cell Sci 113, 3365-3370.
Grosmaire, L., Ledbetter, J., Berenson, R., Law, C.-L. (2000). Ligation of CD2 amplifies anti-CD3 x anti-CD28-mediated ex vivo activation and expansion of T cells. Blood 96, 40b.
Johnstone, R. M., and Ahn, J. (1990). A common mechanism may be involved in the selective loss of plasma membrane functions during reticulocyte maturation, Biomed Biochim Acta 49, S70-5.
Jordan, M., Schallhom, A., and Wurm, F. M. (1996). Transfecting mammalian cells: optimization of critical parameters affecting calcium-phosphate precipitate formation, Nucleic Acids Res 24, 596-601.
Latouche, J. B., and Sadelain, M. (2000). Induction of human cytotoxic T lymphocytes by artificial antigen-presenting cells, Nat Biotechnol 18, 405-9.
Marschang, P., Sodroski, J., Wurzner, R., and Dierich, M. P. (1995). Decay-accelerating factor (CD55) protects human immunodeficiency virus type 1 from inactivation by human complement, Eur J Immunol 25, 285-90.
Melkonian, K. A., Ostermeyer, A. G., Chen, J. Z., Roth, M. G., and Brown, D. A. (1999). Role of lipid modifications in targeting proteins to detergent-resistant membrane rafts. Many raft proteins are acylated, while few are prenylated, J Biol Chem 274, 3910-7.
Pessin, J. E., and Glaser, M. (1980). Budding of Rous sarcoma virus and vesicular stomatitis virus from localized lipid regions in the plasma membrane of chicken embryo fibroblasts, J Biol Chem 255, 9044-50.
Pickl, W. F., Pimentel-Muinos, F. X., and Seed, B. (2001). Lipid rafts and pseudotyping, J Virol 75, 7175-83.
Quah, B., O'Neill, H. C. (2000). The application of dendritic cell-derived exosomes in tumor immunotherapy. Cancer Biother. and Radiopharm. 15, 185-194
Raposo, G., Nijman, H. W., Stoorvogel, W., Liejendekker, R., Harding, C. V., Melief, C. J., Geuze, H. J. (1996). B lymphocytes secrete antigen-presenting vesicles. J. Exp. Med. 183,1161-1172.
Saifuddin, M., Ghassemi, M., Patki, C., Parker, C. J., and Spear, G. T. (1994). Host cell components affect the sensitivity of HIV type 1 to complement-mediated virolysis, AIDS Res Hum Retroviruses 10, 829-37.
Simons, K., Toomre, D. (2000). Lipid rafts and signal transduction, Nature Reviews 1, 31-39.
Sprent, J., Cai, Z., Brunmark, A., Jackson, M. R., and Peterson, P. A. (1997). Constructing artificial antigen-presenting cells from Drosophila cells, Adv Exp Med Biol 417, 249-54.
Thery, C., Boussac, M., Veron, P., Ricciardi-Castagnoli, P., Raposo, G., Garin, J., and Amigorena, S. (2001). Proteomic analysis of dendritic cell-derived exosomes: a secreted subcellular compartment distinct from apoptotic vesicles, J Immunol 166, 7309-18.
Zitvogel, L., Regnault, A., Lozier, A., Wolfers, J., Flament, C., Tenza, D., Ricciardi-Castagnoli, P., Raposo, G., and Amigorena, S. (1998). Eradication of established murine tumors using a novel cell-free vaccine: dendritic cell-derived exosomes, Nat Med 4, 594-600.

1. An antigen presenting membrane vesicle comprising on its surface a composition of either relevant molecules for antigen-specific activation or deactivation of T lymphocytes, characterized in that it is present in the form of an artificially induced lipid vesicle budded from a plasma membrane of a eukaryotic, preferably human, cell, wherein the composition of said relevant molecules for activation or deactivation present on the vesicle surface is adjustable, preferably to meet immunological, histological and/or physiological needs or requirements of a particular application or of a disease and/or of a recipient, independently of blood or tissue cells of a recipient.

2. The vesicle according to claim 1, wherein the relevant molecules for T lymphocyte activation comprise relevant molecules for the provision of a signal 1, and preferably also of a signal 2, for the antigen-specific activation of T lymphocytes.

3. The vesicle according to claim 1, wherein the relevant molecules for T lymphocyte deactivation comprise molecules for the provision of a signal 1 for the antigen-specific anergization or induction of negative regulatory T lymphocytes.

4. The vesicle according to claim 1, which is obtained using genetic engineering techniques.

5. The vesicle according to claim 1, wherein said vesicle contains an anti-CD3-single chain variable fragment and/or a HLA molecule with a bound immunogenic peptide, preferably a peptide derived from a tumor- or virus-associated antigen, for the delivery of a signal 1 to T lymphocytes.

6. The vesicle according to claim 1, wherein said vesicle contains at least one costimulatory molecule and/or at least one adhesion molecule, for the provision of a signal 2 for T lymphocyte activation.

7. The vesicle according to claim 6, wherein at least one costimulatory molecule is selected from the group consisting of CD80, CD86 and CD58.

8. The vesicle according to claim 6, wherein at least one adhesion molecule is selected from the group consisting of CD54 (ICAM-1) and DC-SIGN (CD209).

9. The vesicle according to claim 1, wherein said vesicle contains at least one molecule which provides a signal 1 for the antigen-specific anergization or induction of negative regulatory T lymphocytes.

10. The vesicle according to claim 9, which contains at least one HLA-bound allergen or autoantigen, and, where applicable, in addition at least one receptor transmitting an inhibitory signal to T lymphocytes.

11. The vesicle according to claim 1, wherein at least one of the molecules for the provision of a so called signal 1 and/or at least one of the molecules for costimulation and adhesion is targetable and enrichable in lipid rafts, and preferably is a type I transmembrane protein that possesses a lipid raft targeting sequence at its carboxy-terminal region.

12. A process for the production of an antigen presenting vesicles of claim 1, comprising the following steps:
a) cloning of the relevant molecules for antigen-specific activation or deactivation of T lymphocytes, preferably selected from the group consisting of HLA-molecules, costimulatory molecules, adhesion molecules, and antigenic peptides, and cloning of core proteins or core protein fragments of an enveloped virus, preferably the gag-pol or the gag fragment of Moloney MLV;
b) transfecting at least one expression vector construct for antigen-specific activation or deactivation of T lymphocytes and at least one recombinant viral core protein or core protein fragment into a eukaryotic, preferably human, cell and co-expressing the constructs during incubation of the transfected cell in a cell culture system, under conditions that allow antigen-presenting vesicles to be released from the cell culture system into the supernatant; and
c) after incubation harvesting and purifying the antigen-presenting vesicles from the supernatant.

13. The process according to claim 12, wherein the eukaryotic cell is a 293T cell.

14. The process according to claim 12, wherein the recombinant molecules for the T cell activation as well as the viral core proteins are over-expressed.

15. The process according to claim 12, wherein an expression cassette for a CD5L::CD3scFv::CD14 fragment is used, optionally the expression cassette according to FIG. 1a.

16. The process according to claim 12, wherein the an expression cassette for modified ectodomains of type I transmembrane proteins is used, optionally the expression cassette according to FIG. 1b.

17. The process according to claim 12, wherein an expression cassette for the gag-pol or gag fragment of Moloney MLV according to FIG. 1c is used.

18. A vesicle according to claim 1, produced by the process of claim 12.

19. A pharmaceutical composition, preferably a vaccine, comprising antigen presenting vesicles as defined in claim 1.

20. The pharmaceutical composition according to claim 19, wherein the composition of HLA and peptide molecules present on the vesicle surface is specifically adjusted to a recipient's immunological, histological and/or physiological needs or requirements.

21. The pharmaceutical composition according to claim 19, wherein the composition of HLA and peptide molecules present on the vesicle surface is specifically adjusted to a viral or tumor-associated antigen against which a T lymphocyte response shall be triggered.

22. The pharmaceutical composition according to claim 19, wherein the composition of HLA and peptide molecules present on the vesicle surface is specifically adjusted to an allergen or autoantigen against which induction of anergy or negative regulatory T lymphocytes is triggered.

23-29. (Canceled)

30. A method of treating a tumor, said method comprising administering an antigen presenting membrane vesicle according to claim 1.

31. A method of treating an allergy, said method comprising administering an antigen presenting membrane vesicle according to claim 1.

32. A method of treating an autoimmune disease, said method comprising administering an antigen presenting membrane vesicle according to claim 1.

33. A method of expanding a cell type or tissue in vitro or in vivo, said method comprising contacting the cell type or tissue with an antigen presenting membrane vesicle according to claim 1.

34. The method of claim 33, wherein the cell type is selected from the group consisting of a cell of the immune system, a pluripotent precursor cell, and a stem cell.

35. A method of delivering a transmitter or growth factor to a site of action, said method comprising delivering an antigen presenting membrane vesicle according to claim 1 enriched in or carrying said transmitter or growth factor and allowing said vesicle to target to said site of action.

36. A method of identifying an antigen-specific mammalian cell or receptor-specific ligand, said method comprising contacting candidate mammalian cells or ligands with an antigen presenting membrane vesicle according to claim 1 presenting a multivalent binding reagent of interest; and determining whether said vesicle binds said candidate cell or ligand, whereby binding identifies said candidate cell or ligand as an antigen-specific mammalian cell or receptor-specific ligand.

37. The method of claim 36, wherein said mammalian cell is a T lymphocyte.

38. A method for displaying an interacting element for a cell type, said method comprising displaying said interacting element on an antigen presenting membrane vesicle according to claim 1.

39. The method of claim 38, wherein multiple interacting elements are displayed.

40. The method of claim 39, wherein the multiple interacting elements are co-localized on the vesicle surface.

41. A method for phenotypically or functionally characterizing an immune cell, said method comprising contacting the immune cell with an antigen presenting membrane vesicle according to claim 1.

42. The method of claim 41, wherein the immune cell is from an individual with a suspected immunodeficiency or an individual who is being immuno-monitored.
